(19) United States
(12) Plant Patent Application Publication
(10) Pub. No.: US 2002/0188986 P1
(43) Pub. Date: Dec. 12, 2002

(54) Chrysanthemum plant named 'MOZA SI 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/865,148

(22) Filed: May 24, 2001

(51) Int. Cl. A01H 5/00

(52) U.S. Cl. PLT/298

(57) ABSTRACT

A new and distinct Chrysanthemum plant variety characterized by a pink/white color ray florets, consistent flowering response to short days, blooming consistently after 49 days of short day length, small foliage, long lasting blooms, and consistent natural season flowering habit during late September.

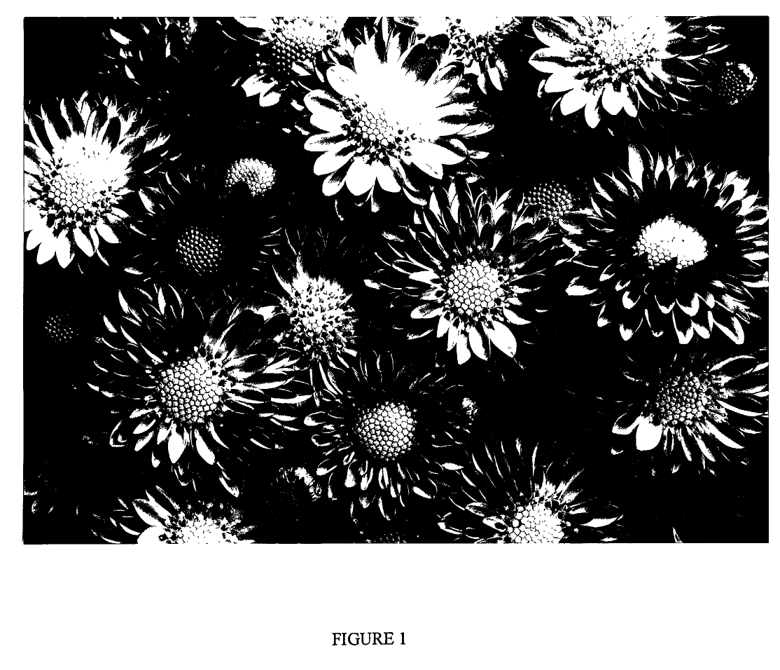

### BACKGROUND OF THE INVENTION

The present invention relates to a new and distinct cultivar of Chrysanthemum plant, botanically known as Dendranthema morifolium and herein after referred to by the cultivar name ‘MOZA SI 01.’

This new cultivar originated from hybridization of a female parent Chrysanthemum cultivar (unpatented in the United States) and a male parent Chrysanthemum cultivar (unpatented in the United States). The new cultivar was discovered by the inventor in November 1998.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE IVENTION

The cultivar ‘MOZA SI 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA SI 01.’ These characteristics in combination distinguish ‘MOZA SI 01’ as a new and distinct Chrysanthemum:

1. Pink/white color ray florets;

2. Consistent flowering response to short days, blooming consistently after 49 days of short day length;

3. Small foliage;

4. Long lasting blooms; and

5. Consistent natural season flowering habit during late September.

Plants of the new cultivar are similar to plants of the female parent cultivar in most horticultural characteristics, however plants of the new cultivar differed from plants of the female parent cultivar in the following characteristics:

1. The new cultivar has different color ray florets;

2. The new cultivar has a smaller diameter bloom;

3. The new cultivar is shorter; and

4. The new cultivar has a shorter internode length.

Plants of the new cultivar are similar to plants of the male parent cultivar in most horticultural characteristics, however plants of the new cultivar differed from plants of the male parent cultivar in the following characteristics:

1. The new cultivar is taller;

2. The new cultivar has a larger bloom diameter; and

3. The new cultivar naturally blooms 2 weeks later.

### BRIEF DESCRIPTION OF THE PHOTOGRAPH

The photograph shows a close up view of the flowers and buds of the new cultivar.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘MOZA SI 01’

Commercial classification: Garden-type Chrysanthemum

Parentage: Hybridization of a female parent Chrysanthemum cultivar (unpatented in the United States) and a male parent Chrysanthemum cultivar (unpatented in the United States).

Propagation

Time to rooting.—7 to 14 days at approximately 21° C.

Rooting habit.—Fine, fibrous

Plant

Growth habit.—Mounding herbaceous perennial

Height.—Approximately 24 cm

Growth rate.—Moderate

Branching characteristics.—Moderate Branching

Length of lateral branches.—Approximately 19 cm

Number of leaves per lateral branch.—Approximately 17

Stem color.—Near 137B

Foliage

Leaf.—Arrangement: Alternate single Average Length: Approximately 5.3 cm Average Width: Approximately 3.2 cm Shape of blade: Ovate Apex: Acuminate Base: Attenuate Attachment: Stalked Margin: Serrate Texture of top surface: Lightly pubescent Texture of bottom surface: Lightly pubescent Color: young foliage upper side: Near 137A young foliage under side: Near 137C mature foliage upper side: Near 137A mature foliage under side: Near 137B venation upper side: Near 138B venation under side: Near 137C

Petiole.—Average Length: Approximately 1.8 cm

Bloom

Inflorescence.—Flowering habit: Induced by darkness period greater than 13.5 hours, approximately 49 days of appropriate day length required to induce and develop blooms Inflorescence form: Daisy Natural flowering season: Approximately the last two weeks in September Number of inflorescences per stem: Approximately 15 Inflorescence diameter: Approximately 3.7 cm Inflorescence depth: Approximately 2.0 cm Inflorescence longevity on plant: Approximately 24-29 days Persistence: Persistent

Ray Florets.—Appearance: Matte Texture: Smooth Average Number per flower: Approximately 240 Shape: Oblanceolate Margin: Entire Apex: Obtuse Length: Approximately 1.8 cm Width: Approximately 0.6 cm Color: upper surface atfirst opening: Near 59C upper surface at maturity: Near 59D at the tip; Near 36D at the base upper surface atfading: Near 73C at the tip; Near 155D at the base under surface at first opening: Near 54A under surface at maturity: Near 63C at the tip; Near 62D at the base under surface at fading: Near 70D at the tip; Near 155D at the base

Disc florets.—Appearance: Shiny Texture: Smooth Average number perflower: Approximately 130 Shape: Cylindric Apex: Obtuse Average Length: Approximately 0.8 cm Average Width: Approximately 0.1 cm Color: at first opening: Near 14B at maturity: Near 15A at fading: Near 17A

Peduncle.—Length: at terminal end (shortest): Approximately 4 cm at lateral end (longest): Approximately 6 cm Strength: Moderate Color: Near 137B Habit: Erect

Inflorescence bud.—Length: Approximately 0.6 cm Diameter: Approximately 0.6 cm Form: Globular Color: Near 54A

Sepals.—Appearance: Matte Texture: Lightly pubescent Number: Approximately 30 to 40 Shape: Ovate Margin: Entire Apex: Acute Color: upper side: Near 137A underside: Near 137A

Reproductive organs

Ray florets.—Pistillate: Number of pistils per flower: 1 Stigma shape: 2 branched Stigma color: Near 10A Style color: Near 8D Style length: Approximately 0.5 cm

Disc florets.—Pistillate: Number ofpistils per flower: 1 Stigma shape: Cylindric Style length: Approximately 0.3 cm Staminate: Number ofstamens perflower: Approximately 5 Anther shape: Tubular Anther color: Near 13A Pollen color: Near 15A

Other Characteristics

Disease resistance.—Neither resistance nor susceptibility observed

‘MOZA SI 01’: Table of R.H.S. Color Codes

| | | | |
|---|---|---|---|
| FOLIAGE | Leaf | young foliage upper surface | Near 137A |
| | | young foliage under surface | Near 137C |
| | | mature foliage upper surface | Near 137A |
| | | mature foliage under surface | Near 137B |
| | | venation upper surface | Near 138B |
| | | venation under surface | Near 137C |
| BLOOM | Ray Florets | upper surface at first opening | Near 59C |
| | | upper surface at maturity, tip | Near 59D |
| | | upper surface at maturity, base | Near 36D |
| | | upper surface at fading, tip | Near 73C |
| | | upper surface at fading, base | Near 155D |
| | | under surface at first opening | Near 54A |
| | | under surface at maturity, tip | Near 63C |
| | | under surface at maturity, base | Near 62D |
| | | under surface at fading, tip | Near 70D |
| | | under surface at fading, base | Near 155D |
| | | Pistillate: Stigma | Near 10A |
| | | Pistillate: Style | Near 8D |
| | Disc Florets | at first opening | Near 14B |
| | | at maturity | Near 15A |
| | | at fading | Near 17A |
| | | Staminate: Anther | Near 13A |
| | | Staminate: Pollen | Near 13A |
| | Peduncle | | Near 137B |
| | Inflorescence | | Near 54A |
| | Bud | | |
| | Sepals | upper surface | Near 137A |
| | | under surface | Near 137A |

What is claimed is:

1. A new and distinct variety of Chrysanthemum plant named ‘MOZA SI 01’ as herein illustrated and described.
